(19) United States
(12) Patent Application Publication
(10) Pub. No.: US 2009/0254155 A1
(43) Pub. Date: Oct. 8, 2009

(54) THERAPEUTIC EMITTER RETAINING DEVICE

(75) Inventors: Maxim Kanarsky, Solon, OH (US); Albert Yakovlevich Grabovschiner, Moscow (RU)

Correspondence Address: RANKIN, HILL & CLARK LLP, 38210 Glenn Avenue, WILLOUGHBY, OH, 44094-7808, US

(73) Assignees: MEDICAL QUANT USA, INC. DBA MULTI RADIANCE MEDICAL, Solon, OH (US); MILTA PKP GIT, Moscow (RU)

(21) Appl. No.: 12/062,899

(22) Filed: Apr. 4, 2008

(51) Int. Cl. A61N 1/00; A61N 5/067

(52) U.S. Cl. 607/89; 607/2

(57) ABSTRACT

A therapeutic device and related method of use are described. The device features a flexible member that includes one or more emitters of for example, electromagnetic radiation, thermal energy, magnetic fields, and/or electrical fields. The device can be readily attached and removed from a user's skin by sealingly contacting the flexible member upon a region of the user's body of interest to create a vacuum. The device may also impart other therapeutically beneficial results such as intensified blood circulation from formation of the vacuum along the user's skin, and can also be used for blood irradiation.

### FIELD OF THE INVENTION

The presently disclosed embodiments are directed to the field of non-invasive therapeutic devices. The embodiments relate to an assembly adapted to affix or otherwise secure one or more low level energy emitting therapeutic elements associated with a therapeutic device or the device itself, to an individual's body. The embodiments include a suction-producing member that readily secures the therapeutic element or device to the individual's body. The embodiments also include a suction-producing member that further promotes the therapeutic effect(s).

### BACKGROUND OF THE INVENTION

A variety of non-invasive therapeutic devices and associated methods are known in the art for treating physiological disorders. Examples of such devices and therapies include, but are not limited to, pain management and relief; tissue repair; bone and joint restoration; biostimulation; and treatment for an array of other disorders and conditions. A common feature of these devices is that they are non-invasive. That is, typically, the devices and related treatment regimes employ the administration of one or more forms of low level energy to the patient, and particularly to the affected area of the patient's body. Examples of the different forms of energy that may be administered or applied include, but are not limited to, electromagnetic radiation, for example light in various forms such as laser light, pulsed light, and/or light at certain wavelengths such as ultraviolet or infrared; thermal energy also referred to as heat; magnetic fields; electrical fields and current; and combinations thereof.

Examples of therapies using emission of electromagnetic radiation or light, include U.S. Pat. No. 5,259,380 to Mendes et al. for a light therapy system. U.S. Pat. No. 5,464,436 to Smith is directed to a method and associated device for performing laser therapy. U.S. Pat. No. 5,405,368 to Eckhouse describes a method and apparatus for therapeutic electromagnetic treatment. U.S. Pat. No. 6,312,451 to Streeter relates to a low level laser therapy apparatus. U.S. Pat. No. 4,671,285 to Walker describes treatment of neurological problems by laser photo stimulation. And, U.S. Pat. No. 4,839,513 to Wijtsma is directed to a device that emits ultraviolet light for treatment of certain skin conditions.

It is well known that surface heat stimulates blood flow to injured tissue. Accordingly, various devices and therapies have been devised to administer heat to an affected area of an individual's body. For example, U.S. Pat. No. 5,358,503 to Bertwell et al. is directed to a device which emits light and thermal energy, and is adapted to be held alongside the affected area.

The application of magnetic fields to a patient's body for therapeutic reasons is also known in the art, such as shown in U.S. Pat. No. 6,641,520 to Bailey et al. U.S. Pat. No. 4,501,265 to Pescatore is directed to an applicator head for electromagnetic treatment of an afflicted body region.

And, the application of electrical fields and/or administration of an electrical current to an individual's body, or region thereof, is also known in the art. U.S. Pat. No. 4,023,574 to Nemec; U.S. Pat. No. 4,580,570 to Sarrell et al.; and U.S. Pat. No. 4,989,605 to Rossen all relate to such therapies.

In all of the previously noted treatment strategies, the devices that emit the particular form of energy, are typically held in position by the user or by an individual providing the therapy. In certain instances, a temporary means of affixment may be used such as an elastic band as noted in the previously described U.S. Pat. No. 5,358,503 to Bertwell et al.

Although satisfactory in certain respects, the use of elastic bands or other conventional attachments such as adhesives, adhesive pads, Velcro, and/or flexible straps is undesirable. Adhesives or pads containing such often leave a residue on the patient's skin. In addition, removing adhesively attached members can result in pain or at least significant discomfort as the member is pulled from the wearer's skin. Elastic bands, Velcro, and/or straps are cumbersome and can irritate the affected area of the wearer. In addition, if formed from a cloth or other absorbent material, such items can become dirty or otherwise contaminated, thus increasing the likelihood of infection if the affected area includes an open skin lesion or abrasion.

Accordingly, a need exists in the art for a member that includes one or more non-invasive therapeutic elements that can be easily secured to and removed from an individual's body, and in particular, held alongside an affected region of the body in a hands-free manner.

The use of suction cups and suction-producing devices in medicine are known. More specifically, suction cups and vacuum-producing devices are known for treating a wide array of ailments and conditions. For example, U.S. Pat. No. 4,287,819 to Emerit describes a device for placing over an injured area and creating a partial vacuum over that area. Emerit describes his device as useful for withdrawing venom from a bite or sting. U.S. Pat. No. 5,308,321 to Castro describes a cup-shaped member that imparts a vacuum that is used in conjunction with a breast examination procedure. U.S. Pat. No. 7,229,422 to Klobe is directed to a suction cup for use in a non-surgical procedure to treat a sunken chest. Although directed to a range of different disorders and conditions, none of these devices and approaches relate to non-invasive therapies using low level energy emission to an affected area of an individual. And, none of these patents describe the incorporation of one or more energy emitters in a suction cup or suction-producing member.

However, artisans have devised suction cups that include a component which is used in a medical or therapeutic treatment regime. For example, U.S. Pat. No. 6,733,493 to Gruzdev et al. describes a laser skin perforator. The device features a relatively high powered laser that is used to cut or perforate the skin, and thus is an invasive device. The device uses a retaining arrangement that is said to intensify blood circulation in the skin area that is selected for perforation. U.S. Pat. No. 4,134,395 to Davis is directed to diagnostic screening examinations using high intensity magnetic fields. In one embodiment, Davis describes an electromagnet that can be attached to a wearer's body using a suction cup in order to secure the magnet near an organ of interest while a high powered magnetic field is applied. Non-invasive strategies of attaching electrodes to a patient's body, are described in U.S. Pat. No. 5,345,935 to Hirsch et al. and U.S. Pat. No. 6,456,865 to Samson. The patents both describe the use of suction cups that are disposed around the electrodes, and which serve to attach the electrode to a person's body. The suction cups are configured to place the electrode in contact with the person's skin for receiving electrical signals at the electrode. Although satisfactory in many respects, these various devices and strategies are not applicable to non-invasive therapies using low levels of energy emissions such as light, heat, and electricity.

Therefore, in view of the foregoing, a need exists for a member that includes an emitter of relatively low level energy adapted for non-invasive therapeutic applications, and which can be readily attached to an individual's body by forming and maintaining a partial vacuum along the body region of interest.

Artisans have recognized benefits from application of a vacuum to an individual's skin. The previously mentioned patent, U.S. Pat. No. 6,733,493 to Gruzdev et al. describes a laser skin perforator. Although the device employs a relatively high powered laser for cutting or perforating the skin, the device uses a retaining arrangement for intensifying blood circulation in the skin area that is selected for perforation. This physiological effect is sometimes referred to as “cupping.” U.S. Pat. No. 5,662,677 to Wimmer describes a cupping instrument. However, that device is merely used to create an area of reduced pressure along a person's skin.

As far as is known, artisans have not recognized the benefits of intensifying blood circulation in a body region in conjunction with applying a therapeutic low level energy to that region. Accordingly, a need exists for a device and related method in which blood circulation in a region of a person's body is intensified while concurrently applying a therapeutic low level energy to that region.

Blood irradiation is a process by which certain agents such as pathogens, are killed or otherwise rendered harmless, by irradiating the blood. A variety of techniques are used to effect irradiation such as exposure to gamma radiation, nuclear radiation and light radiation. Regarding the use of light, ultraviolet blood irradiation is an example of such and first evolved in the 1930's as a means to treat people afflicted with the poliovirus. Most notably, this was reported in an article entitled “UBI”, which was published in TIME, on Jun. 13, 1949. This process has been resurrected, and various treatment strategies have been described using this approach. See for example, US Patent Publications 2004/0039325 and 2006/0270960, both to Karp. Karp provides a detailed history of ultraviolet blood irradiation, and describes a device and related strategy by which blood is removed from the patient, and then exposed to specific light emissions. An in-vivo strategy for irradiating blood by ultraviolet light is described in U.S. Pat. No. 5,693,049 to Mersch.

Although apparently satisfactory, the noted previous approaches to blood irradiation via light have relied upon exposing the blood to ultraviolet light after the blood has been removed from the body. The noted in-vivo strategy required invasive catheters to be utilized through which blood flowed past various light emissions. Understandably, it is undesirable to withdraw blood from a patient due to the risk of infection and other concerns in addition to the discomfort associated with perforating the patient's skin. Accordingly, it would be desirable if blood irradiation could be performed in a non-invasive manner such that the blood is not removed from the patient.

### SUMMARY OF THE INVENTION

The difficulties and drawbacks associated with previous-type systems are overcome in the present method and apparatus for a vacuum-producing retaining device, which enables a person to secure a therapeutic device or element to the person's body, without cumbersome straps, buckles, adhesives, or other affixment means typically used in the medical arts. In certain versions, the vacuum-producing retaining device can be used to intensify blood circulation in a region of a person's body while also administering a therapeutically effective amount of energy to that region. And, certain versions of the device can be utilized for performing non-invasive blood irradiation procedures.

In a first aspect, the present invention provides a non-invasive therapeutic device adapted for selective attachment to and removal from a surface region of an individual's body. The device comprises a flexible member defining a medial portion and a wall extending outwardly therefrom. The wall and medial portion define a hollow interior accessible from a face of the flexible member. The wall defines a distal edge that upon attachment of the device to the body region, sealingly contacts the surface region. The medial portion and the wall both define an inner surface facing the hollow interior of the flexible member. The device also comprises at least one element adapted to emit a therapeutically effective level of energy. The element is affixed to the inner surface of the flexible member.

In another aspect, the present invention provides a method for selectively attaching a non-invasive therapeutic device to a surface region of an individual's body. The method comprises providing a non-invasive therapeutic device including (i) a flexible member defining a medial portion and a wall extending outwardly therefrom. The wall and medial portion define a hollow interior accessible from a face of the flexible member. The wall defines a distal edge. The device also includes (ii) at least one element disposed in and affixed to the flexible member and adapted to emit a therapeutically effective level of energy. The method also comprises contacting the distal edge of the wall of the flexible member to the surface region of the individual's body and creating a vacuum in the hollow interior of the flexible member, thereby attaching the device to the surface region of the body.

In yet another aspect of the present invention, a method is provided for intensifying blood circulation in a body region while concurrently applying a therapeutically effective level of energy to that region. The method comprises providing a non-invasive therapeutic device adapted for selective attachment to and removal from a surface region of an individual's body. The device comprises (i) a flexible member defining a medial portion and a wall extending outwardly therefrom. The wall and the medial portion define a hollow interior accessible from a face of the flexible member. The wall defines a distal edge that upon attachment of the device to the body region, sealingly contacts the surface region. The medial portion and the wall both define an inner surface facing the hollow interior of the flexible member. The device also comprises (ii) at least one element adapted to emit a therapeutically effective level of energy. The element is affixed to the inner surface of the flexible member. The method also includes attaching the device to a surface region of an individual's body by sealingly contacting the distal edge of the wall to the surface region and creating a vacuum in the hollow interior of the flexible member, thereby intensifying blood circulation in the body region. And, the method includes emitting a therapeutic level of energy to the body region while the device is attached to the body region.

It is an object of the present invention to provide a device or device component that includes one or more non-invasive therapeutic elements that can be easily secured to and removed from an individual's body, and in particular, alongside an affected region of the body, without the associated disadvantages of previously known retention strategies such as by using elastic bands, adhesives, adhesive pads, Velcro, and/or flexible straps.

It is another object of the invention to provide a device or device component that includes an emitter of relatively low level energy adapted for non-invasive therapeutic applications, which can be readily attached to an individual's body by forming and maintaining a vacuum along the body region of interest.

It is another object of the invention to provide a device and related method in which blood circulation in a region of an individual's body is intensified, while concurrently applying a therapeutic low level energy to that region.

It is also another object of the present invention to provide a device and related method for irradiating blood in a non-invasive manner.

As will be realized, the invention is capable of other and different embodiments and its several details are capable of modifications in various respects, all without departing from the invention. Accordingly, the drawings and description are to be regarded as illustrative and not restrictive.

### BRIEF DESCRIPTION OF THE DRAWINGS

FIG. 1 shows the general configuration of a preferred embodiment device of the present invention.

FIG. 2 shows the underside or contacting face of the preferred embodiment device depicted in FIG. 1.

FIG. 3 depicts the preferred embodiment device in use, and positioned upon an affected region of a user's arm.

FIG. 4 is a schematic view of the preferred embodiment device initially placed upon a user's arm.

FIG. 5 is a schematic view of a compressive force applied to the preferred embodiment device on the user's arm to partially evacuate the hollow interior of the device.

FIG. 6 is a schematic view of the preferred embodiment device on the user's arm, after the device resiliently returns to its original shape to thereby create a vacuum within the hollow interior.

FIG. 7 is a schematic partial view of another preferred embodiment device, illustrating an alternative configuration for the edge of a flexible member of the device.

FIG. 8 is a schematic partial cross sectional view of the preferred embodiment device depicted in FIG. 7, placed upon and contacting a user.

FIG. 9 is a schematic partial view of another preferred embodiment device, illustrating an alternative configuration for the edge of a flexible member of the device.

FIG. 10 is a schematic partial cross sectional view of the preferred embodiment device depicted in FIG. 9, placed upon and contacting a user.

FIG. 11 shows the general configuration of another preferred embodiment device of the present invention.

FIG. 12 shows the underside or contacting face of the preferred embodiment device depicted in FIG. 11.

FIG. 13 shows the general configuration of another preferred embodiment device of the present invention.

FIG. 14 shows the underside or contacting face of the preferred embodiment device depicted in FIG. 13.

FIG. 15 is a schematic illustration of application of a flexible sealing member and irradiation of a body region.

FIG. 16 is a schematic illustration of irradiation of a body region without a vacuum.

FIG. 17 depicts another preferred embodiment device in use, and positioned upon an affected region of a user's arm.

### DETAILED DESCRIPTION OF THE EMBODIMENTS

The present invention provides a variety of preferred embodiments of devices that comprise a flexible sealing member and one or more energy emission elements generally disposed and incorporated within the sealing member. The energy emission elements provide therapeutically effective levels of one or more forms of energy for therapeutic uses as described herein. The sealing member generally includes a medial portion and an outwardly extending wall that projects from the medial portion. Typically, the wall is arcuate in shape and preferably, circumferential so as to define a cylindrical, semi-cylindrical, or cone-shaped closed interior hollow region. Depending upon the configuration of the wall, the distal edge of the wall may take a variety of forms. For embodiments in which the wall is circumferential, the distal edge defines a circular shape. The shape of the wall may be any shape which enables the wall to still be flexible. However, preferred shapes for the sealing member include cup-shaped and cone-shaped.

It is instructive to further explain several terms that are used herein. The term “therapeutically effective” is used herein to refer to an amount or level of energy that is used in administering a therapeutic treatment. Thus, that term refers to an energy level that is typically used in the therapy of interest, and as such, is an amount or level that imparts therapeutic benefits to the affected body area, and is less than an amount or level that would cause immediate or severe damage to the individual and/or tissue.

The term “vacuum” as used herein refers to a pressure that is less than atmospheric pressure. Thus, the term vacuum refers to a pressure of 0.5, 0.7, 0.8, or 0.9 atmospheres for example. It will be appreciated that the term refers to any pressure less than atmospheric pressure which is typically 1.0 atmosphere. Generally, the term refers to a pressure of from about 0.1 to about 0.99 atmospheres, typically from about 0.2 to about 0.95 atmospheres, and preferably from about 0.3 to about 0.90 atmospheres. Most preferably, the flexible sealing members described herein achieve vacuums of from about 0.48 atmospheres to about 0.68 atmospheres, (0.5 to 0.7 kg/cm²).

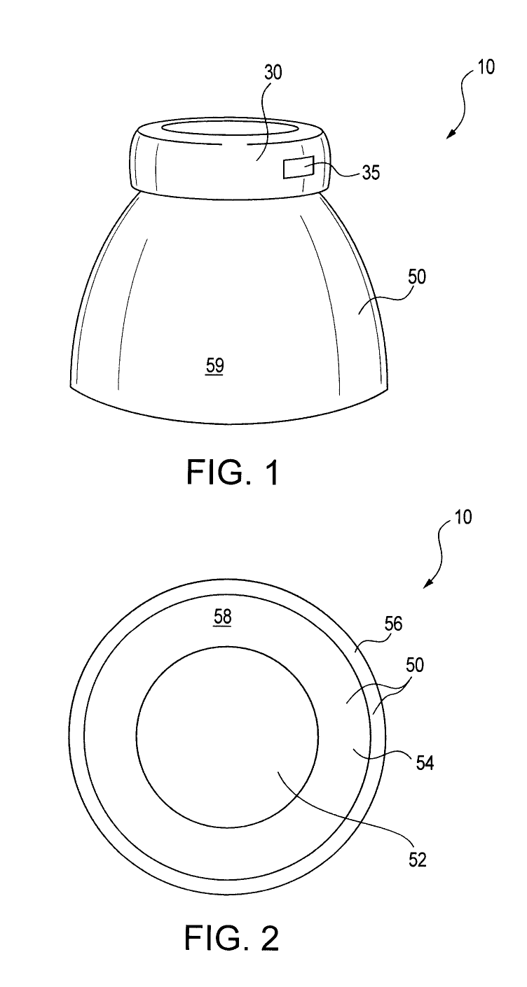

FIGS. 1 and 2 illustrate a preferred embodiment device 10 in accordance with the present invention. The device 10 comprises an energy emitting element, combination of such elements, and/or a housing enclosing such, generally represented as item 30. The energy emitting element may include provisions for connection to an external unit, by use of a cable port 35. The device 10 also comprises a flexible member 50. Preferably, the flexible member 50 is integrally formed or attached with the element(s) or housing 30. The flexible member 50 includes a medial portion 52 and a wall or plurality of walls shown as 54. The wall 54 extends outward from the medial portion 52 and defines a distal edge 56. The flexible member 50 and the medial portion 52 define an interior surface 58, and an oppositely directed outer surface 59.

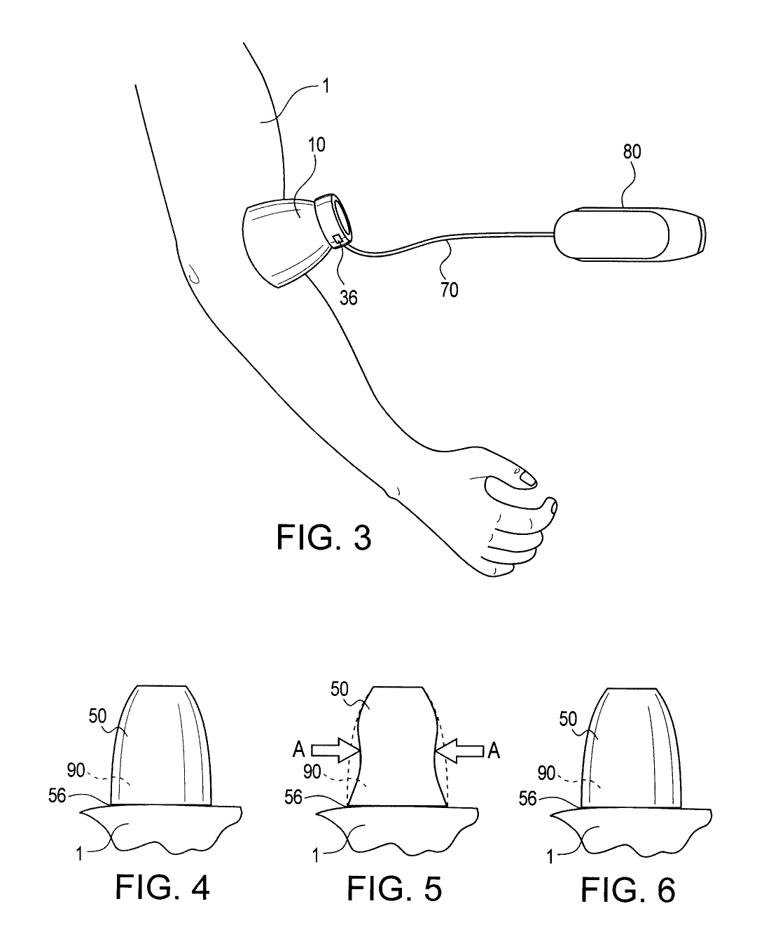

FIG. 3 illustrates the preferred embodiment device 10 in use, and positioned upon an individual's arm 1. The device 10 can be electrically connected to a base unit or instrument 80 by use of a cable 70. Cable 70 attaches to the device 10 via cable port 35 shown in FIG. 1. An optional interface port 36 may also be provided.

FIGS. 4-6 schematically illustrate placement, sealing, and creation of a vacuum within the hollow interior of the device, as the device is positioned upon an individual's body. Specifically, FIG. 4 shows the flexible member 50 of the device positioned upon a person's arm 1, such that the distal edge of the wall 56 is in contact with the arm 1. The hollow interior of the flexible member 50 is depicted as item 90. FIG. 5 illustrates a compressive force A applied to the sides of the flexible member to thereby reduce the volume of the interior 90. During this compression, air is typically expelled from the interior 90 between the distal edge 56 and the arm 1. Once the evacuated air has been expelled, and a sealing contact is attained between the distal edge 56 and the arm 1, the resilient nature of the flexible member 50 causes that member to return to its original shape, thereby restoring the interior 90 to its original volume. This in turn creates a reduction in the pressure within the interior, and thus, creates a vacuum within the interior 90. It will be understood that the device 10 can be attached to nearly any region of a user's body besides an arm. A significant feature of the flexible sealing members described herein and their use, is that they provide secure attachment of the device in a desired region of the individual's body or skin as a result of the vacuum maintained at that region.

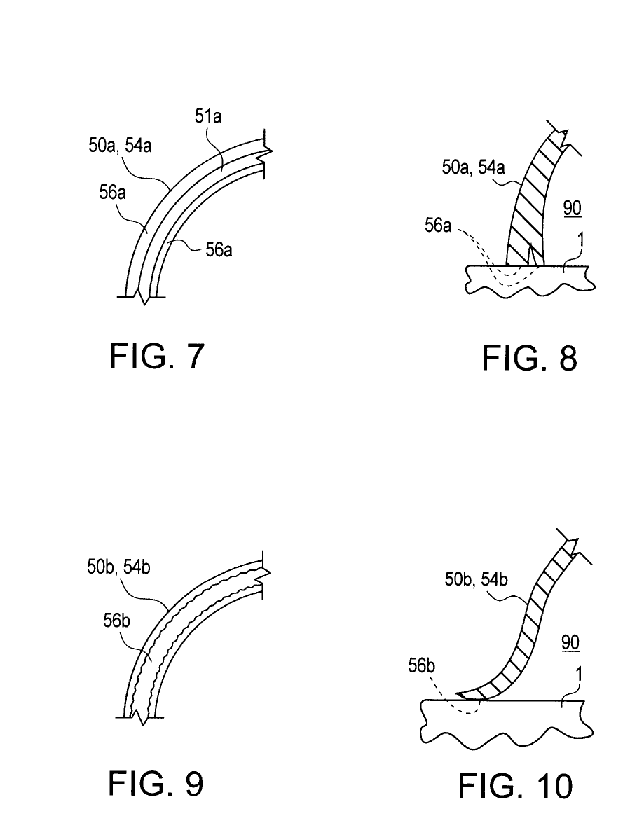

The present invention includes a wide variety of different configurations for the flexible sealing member and details such as shape and manner in which the member contacts or engages a user's skin to achieve a seal sufficient to maintain a vacuum within the flexible member. For example, FIGS. 7 and 8 illustrate a preferred embodiment distal edge configuration wherein the edge designated as item 56a, includes an outer circumferential portion and an inner circumferential portion. A small void or space 51a is defined between the two portions, and promotes sealing by allowing the inner portion to be relatively flexible and adaptive to any irregularities or curvature in the underlying surface to be sealed against, i.e. the user's body. The walls of the flexible member are designated as 54a and 50a, respectively. Similarly, FIGS. 9 and 10 depict another preferred variant for the distal edge of the flexible wall member, shown as edge 56b. In this configuration, the edge 56b does not extend toward the user's body, but instead, extends alongside in a tangential fashion. The interior formed within the walls 54b and 50b of the flexible member, and an arm 1, is denoted as 90. This configuration may be desirable for certain applications and features a relatively large sealing surface without a corresponding increase in wall thickness.

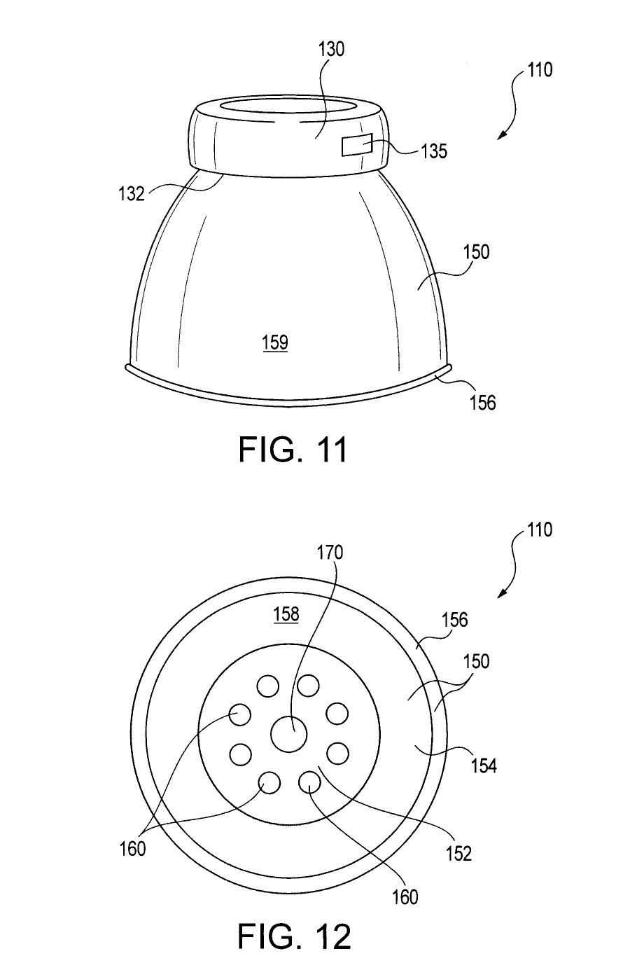

FIGS. 11 and 12 illustrate another preferred embodiment device 110 in accordance with the present invention. The device 110 comprises an energy emitting element, combination of such elements, and/or a housing enclosing such, generally represented as item 130. The energy emitting element may include provisions for connection to an external unit, by use of a cable port 135. The device 110 also comprises a flexible member 150. Preferably, the flexible member 150 is integrally formed or attached with the element(s) or housing 130 such as for example, along an interface 132. The flexible member 150 includes a medial portion 152 and a wall or plurality of walls shown as 154. The wall 154 extends outward from the medial portion 152 and defines a distal edge 156. The flexible member 150 and the medial portion 152 define an interior surface 158, and an oppositely directed outer surface 159.

As particularly shown in FIG. 12, the preferred embodiment device 110 shown in FIGS. 11 and 12 features a combination of energy emitting elements 160 and 170. Preferably, the elements 160 are light emitting diodes, and the centrally disposed element 170 is a laser emitting element. Although the configuration of energy emitting elements 160 and 170 shown in FIG. 12 employs a plurality of light emitting diodes 160 disposed around a centrally disposed laser emitting element 170, the present invention includes other arrangements and configurations of energy emitting elements.

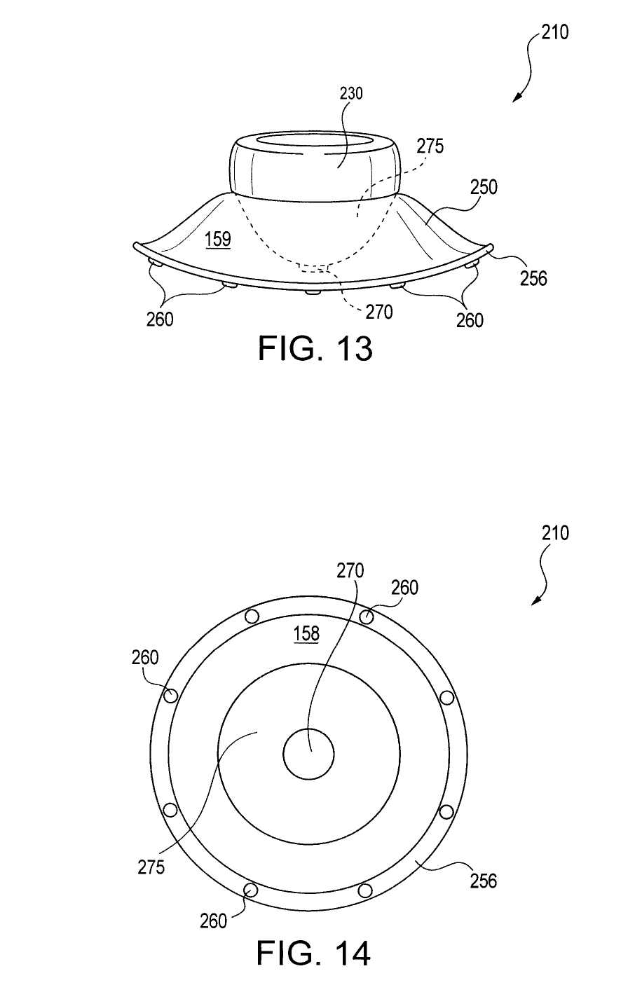

FIGS. 13 and 14 illustrate another preferred embodiment device in accordance with the present invention. FIGS. 13 and 14 illustrate a device 210 adapted to place one or more electrodes in contact with a user's skin. Examples of such electrodes are those that administer an electrical current or voltage across a user's skin or body region of interest. It is to be noted that the one or more electrodes are to be distinguished from prior art electrodes that are conventionally placed upon a user's skin to receive electrical signals in order to monitor the patient. The preferred embodiment device 210 includes a housing 230 that includes the one or more energy emitting elements and the electronics for the one or more electrodes. Extending from the housing 230 is a flexible member and wall 250, generally as previously described with regard to the preferred embodiment devices 110 and 10. The flexible member defines an exterior surface 159 and an oppositely directed interior surface 158. It will be understood that the device 210 may feature a shorter profile than the previously described devices 110, 10 as a result of the interior configuration of the device 210. Preferably, the device 210 includes an outwardly projecting and centrally disposed member 275, which provides one or more electrodes 270. The member 275 extends to a degree or depth such that the electrode 270 is placed in electrical contact with the user, upon placement of the device 210 upon the user's body region. Another significant feature of the preferred device 210 is that one or more electrodes 260 may be provided along the distal edge 256 of the wall 250. These electrodes can be used instead of, or in addition the electrode 270. The one or more electrodes 260, 270 are adapted to provide an electrical current or potential at their surfaces for imparting an electrical current or potential at or along a user's skin, such as for example, in accordance with a TENS therapeutic procedure. It is contemplated that one or more agents could be used on the exposed electrode surfaces to promote electrical conductivity between the electrode and the user's skin. Furthermore, it is also contemplated to configure the use of the electrodes 260, 270 so that specific electrical patterns may be discharged or otherwise produced from the collection of electrodes. Time varying electrical voltage patterns discharged from the electrodes may be administered.

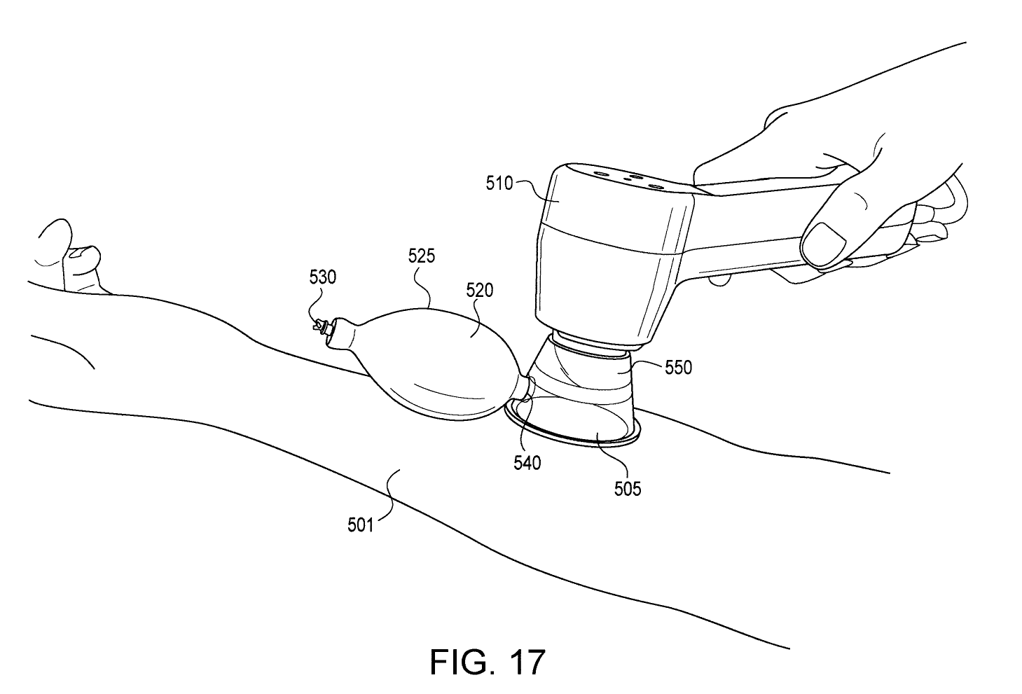

FIG. 17 illustrates another preferred embodiment device 510 and flexible sealing member 550 in accordance with the present invention. In this embodiment, a vacuum producing bulb 520 is in flow communication with the interior region of the sealing member 550. An access port 540 defined in the side wall of the member 550 can be provided to provide such access. The bulb 520 is provided with a flexible deformable hollow member 525 and an air valve 530 for creating and maintaining a vacuum within the member 525 and thus also within the sealing member 550. The device 510 and bulb 520 is used by placement of the member 550 on a body region 501 and specifically, in close proximity to a region 505 of interest. After placement of the distal edge of the member 550 on the region 505, the bulb 520 is compressed thereby forcing air out of the valve 530. The resilient nature of the bulb 520 causes it to return to its original shape and volume, or substantially so, and thereby create and maintain a vacuum over the area 505.

The flexible sealing member, and particularly the wall of the sealing member, can be formed from a variety of materials. Preferably, the material(s) selected are those that enable the wall to be flexible so that the wall and particularly, the distal edge of the wall, can sealingly contact a user's body or skin. A wide array of materials can be used for the sealing member and particularly the wall, such as for example thermoplastics and thermoplastic elastomers (TPEs). Depending on the hardness, TPEs are sometimes categorized as thermoplastics and sometimes as elastomers. For purposes of this invention, no such distinction will be made, and hard and soft grades of plastic will all be referred to as TPEs. TPEs are commercially available in several different brands and types. Each type can be obtained in different grades having different properties such as, hardness, tensile strength, compression, elongation, thermal stability and colorability. Selection of the appropriate TPE for a particular application depends on a suitable combination for such properties. Types of TPEs which are particularly useful are styrenic block co-polymers, rubber polyolefin blends, elastomeric alloys, thermoplastic alloys, thermoplastic elastomeric alloys, thermoplastic isomers, thermoplastic polyurethanes, polyvinyl chlorides and blends thereof. Styrenic block copolymers are commercially available in many types (and grades within types). The rubber-polyolefin blends (or thermoplastic polyolefins (TPOs)) are blends of various polyolefins with ethylene-propylene rubber (EPR) or ethylene-propylene-diene-monomer (EPDM). Suitable polyolefins include polypropylene and various types of polyethylene. Copolymers of propylene and ethylene and blends of TPOs can also be used. TPOs are also useful as modifiers of other TPEs. Thermoplastic polyurethanes (TPUs) are formed by copolymerization of diisocyanates with long-chain diols and short-chain diols. TPUs are available commercially in a number of types and grades. Polyvinyl chloride (PVC) based TPEs are also suitable for the sealing member and are available in different grades and blends with other TPEs and rubbers.

Alloying is an interactive combination of two or more materials to give a material having better properties than those of the corresponding blend. Thermoplastic alloys are available with properties enabling them to be flexible and suitable for the present invention. Thermoplastic elastomeric alloys and elastomeric alloys (EAs) are composed of synergistic mixtures of two or more polymers that have been treated to give them properties different from simple blends of the major constituents. The two types of elastomeric alloys are melt processable rubbers (MPRS) and thermoplastic vulcanizates (TPVs).

Preferably, the flexible member is manufactured from a soft elastomeric material, such as PVC, or other thermal plastic or liquid injection molded material, such as silicone, but it is also possible to manufacture it from other suitable elastomeric materials. Additional preferred materials for forming the flexible member include rubber and latex materials. The flexible member can be impregnated with anti-microbial agents to promote and maintain a relatively bacteria-free surface. In certain embodiments, the flexible member can be disposable to facilitate sanitary and hygienic purposes. The particular wall thickness of the flexible member can be adjusted to facilitate desired flexing characteristics and rigidity.

An important characteristic of the material selected for the flexible member, and particularly for the wall of the flexible member, is that the material be resilient and somewhat elastic. That is, it is preferred that after compression of the flexible member to thereby reduce the volume of the interior of the member, the resilient characteristic of the material causes the member, and particularly the wall of the member, to return to its previous shape and configuration prior to the compression. Thus, after release of a compressive force causing compression, it is preferred that the flexible member readily returns to its original shape.

The flexible member of the present invention device can be of any size so as to promote a sealing contact between the distal edge of the member and the body region of interest. For distal edges of walls of flexible members that are circular, typical diameters are from about 2.5 cm (1 inch) to about 10 cm (4 inches). Specifically for example, the diameter of the base should not be more than about 2.5 cm to about 3 cm for the treatment of small joints. For the treatment of cellulite, the diameter of the base should not be more than about 5 cm to about 6 cm. The present invention includes smaller or larger sizes, and different shapes for the flexible member. As will be appreciated, the present invention includes any shape for the flexible member.

The present invention also includes embodiments in which a device uses a flexible member that is readily detachable from a housing. For example, referring to FIG. 11, the flexible member 150 is detachable from a housing 130, generally along the interface 132. This configuration facilitates the use of disposable flexible members. Thus, after use of the device, the flexible member having previously contacted a user's body, could then be easily removed from the housing and discarded or collected for subsequent cleaning or recycling. For applications in which a detachable flexible member is not necessary, the member could be cleaned and/or sterilized in accordance with conventional clinical practices.

As noted, the device also includes one or more energy emitting elements. Preferably, these energy emitting elements are disposed within the sealing member and are positioned and oriented such that the emitted energy is directed out of the sealing member. Preferably, the one or more energy emitting elements are incorporated within the noted medial portion or member of the flexible sealing member, and are directed such that, upon operation, their respective emission(s) are directed out through the face or opening of the flexible member.

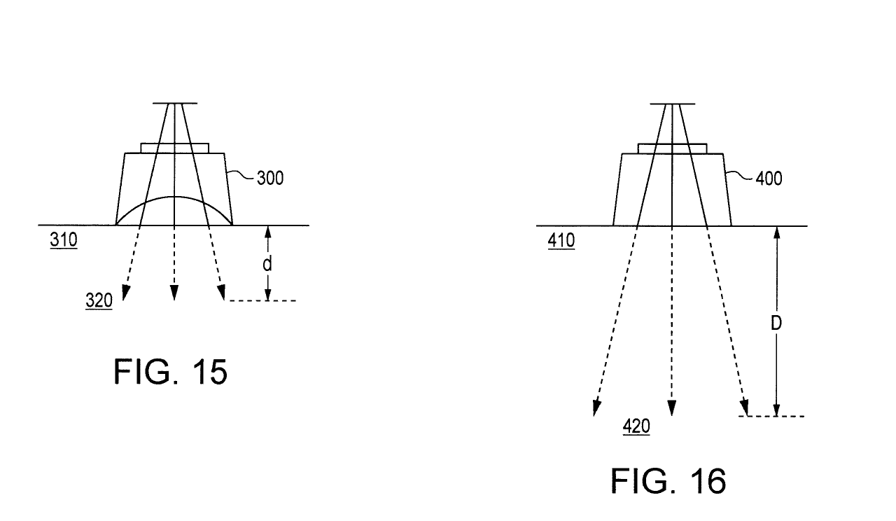

As explained herein, by use of the flexible sealing member, blood and other body fluids can be brought closer to the surface of a body region, at which the blood and/or other fluids can be exposed to one or more forms of energy as described herein. FIG. 15 is a schematic illustration of absorption of laser and infrared radiation 320 in a body region 310 for example, during application of a vacuum by a sealing member 300 as described herein. Radiation 320 travels to a depth d in the region of increased blood flow. FIG. 16 illustrates passing through of laser and infrared radiation 420 in a body region 410 to a greater depth D, in the absence of a vacuum. Items 300 and 400 in FIGS. 15 and 16 represent preferred embodiment devices having flexible sealing members and provisions for emitting radiant energy as described herein.

The present invention also includes embodiments in which the inner surface of the flexible sealing member includes a reflective material. The use of a reflective material along the inner surface or portion of the inner surface of the flexible member can promote the administration of energy, particularly electromagnetic radiation or light, from the respective element(s) within the flexible member to the body region of interest.

The energy emitting elements can be of any type as are typically used in therapeutic devices as described herein. Thus, examples of such elements are those that emit electromagnetic radiation, for instance light in various forms such as laser light, pulsed light, and/or light at certain wavelengths such as ultraviolet or infrared; thermal energy also referred to as heat; magnetic fields; electrical fields and current; and combinations thereof. Regarding light emitting elements, it is preferred to use one or more elements that emit certain wavelengths of light to achieve various colors, such as for example blue, red, and infrared. Table 1, set forth below, lists representative ranges of wavelengths for light emitting elements for use in the present invention.

TABLE 1
Wavelength Ranges

| | Wavelength Range (nm) |
|---|---|
| Infrared | |
| Long Wavelength IR | 8,000-15,000 |
| Medium Wavelength IR | 3,000-8,000 |
| Short Wavelength IR | 1400-3000 |
| Near IR | 750-1400 |
| Visible | |
| Red | 620-750 |
| Orange | 590-620 |
| Yellow | 570-590 |
| Green | 495-570 |
| Blue | 450-495 |
| Violet | 380-450 |
| Ultraviolet | |
| Near UV | 400-200 |
| UVA, Long | 400-320 |
| UVB, Medium | 320-280 |
| UVC, Short | below 280 |
| Far or Vacuum UV | 200-10 |

It is particularly preferred for certain applications to utilize light emitting elements that emit light within a wavelength range of from about 600 nm to about 910 nm. Particularly preferred ranges of wavelengths for emission are those at or around 630 nm, 660 nm, 700 nm, 800 nm, 875 nm, and 905 nm. Without being limited to any particular theory, these wavelengths are typically those for which biological tissues are responsive. Another factor which can dictate the particular frequency of light selected, is the depth of tissue that is being targeted. Generally, the depth of penetration increases by using light of a longer wavelength. Thus, relatively deep penetrations of tissue can be attained by using light emissions of relatively long wavelengths, such as from about 900 to about 1,000 nm.

The light emitting elements can be in nearly any form known in the art. For example, light emitting elements such as light emitting diodes are preferred. However, other types of light emitting elements can be used such as incandescent elements, arc elements, gas discharge elements (e.g. fluorescent, neon, argon, xenon lamps) and others.

Regarding a laser light emitting element, it will be understood that the term “laser” is an acronym: Light Amplification by Stimulated Emission of Radiation. A typical laser emits light in a narrow, low-divergence beam and with a well-defined wavelength corresponding to a particular color if the laser is operating in the visible spectrum. The present invention device can employ one or more laser(s) that emit light at a particular wavelength or narrow band, or a narrow band within any of the noted wavelength ranges set forth in Table 1. It is preferred for certain applications to utilize a laser at a wavelength of 905 nm.

The present invention also includes the use of one or more energy emitting elements that emit light in a pulse fashion. That is, light at any of the noted ranges of wavelengths may be emitted in a sequential series of intermittent bursts or “pulses”. The pulses of light are characterized by (i) the pulse repetition time or number of pulses per unit time, and (ii) the pulse width or pulse duration. The wavelength of the pulsed light can be selected from those set forth in Table 1, preferably those in the visible region. The present invention can employ a wide array of pulse patterns in administering a therapeutically effective treatment program. Typically, pulse width or pulse duration depends upon the particular treatment strategy and the power level of the emission(s). For most biological systems, pulse durations are preferred which are at least about 1 millisecond up to about 1 second. However, for certain treatments, it may be preferred to employ significantly shorter pulse durations such as if using significantly higher power or energy levels. For example, for high power laser emissions, it may be preferred to utilize pulse durations on the order of from about 100 to about 200 nanoseconds. However, it is to be appreciated that the present invention includes devices utilizing one or more light emitting elements that emit light in a continuous mode upon activation. Specifically, the present invention includes the use of a laser configured to emit laser light in a continuous fashion upon actuation.

The present invention includes the use of energy emitting elements that are adapted to emit thermal energy to the body region of interest. For devices of the present invention including one or more of these elements, it is contemplated that they be operated so as to achieve a skin surface temperature of from about 36° C. to about 41° C. It will be understood that many of the light emitting elements such as certain infrared emitting elements described herein may also serve as thermal emission elements.

For magnetic field emitting elements, it is generally preferred to use those that emit or otherwise produce low levels of such fields, such as less than 500 gauss, and preferably less than 300 gauss. Without being limited to any specific theory, it is contemplated that these lower levels of magnetic fields are preferred for administering therapeutic procedures as described herein, as opposed to the use of higher gaussian levels. Higher levels are typically used in magnetic imaging procedures, such as magnetic fields of 500 to 10,000 gauss.

It is instructive to consider the terminology used herein with regard to the energy emitting elements. An electric field is a field or force that exists in the space between two different electrical potentials or voltages. An electric field is also known as an electrostatic field. As will be appreciated, an electrical field can produce an electrical current, and a current can produce an electric field. A moving charge or current, has in addition to an electric field, a corresponding magnetic field. In general, the electric and magnetic fields are not completely separate phenomena. However, these various terms are used herein to refer to the primary effect of the energy emitting element. Therefore for example, a light emitting diode (LED) is considered for purposes herein as a light emitting element even though it generates a relatively small amount of heat. If an element primarily generates a magnetic field, and a secondary electric field, for purposes of the present description, that element is considered herein as a magnetic field emitting element. And vice versa, if an element primarily generates an electric field, and only a slight magnetic field, that element is designated herein as an electric field emitting element. Similarly, an element adapted to induce or otherwise cause an electrical current or intermittent current or charge across the skin or below the skin, such as a Transcutaneous Electrical Nerve Stimulation (TENS) device, is considered herein as an electrical current emitting element rather than an electric field generating element even though an electric field may be generated during its use. It will be understood that the present invention is not directed to new developments relating to the various energy emitting elements, themselves. Instead, the present invention provides a new and beneficial device and strategy for using, combining and/or configuring those elements such that they may be easily and readily attached or otherwise secured to a user's body, used in certain fashions, and maintained in a desired relation with the user's body. Accordingly, no further details are provided herein of the details of the various energy emitting elements. Also, these various elements are commercially available.

For certain applications, it is particularly preferred to provide combinations of different types of the energy emitting elements in the device(s). For example, combinations of one or more different light emitting elements can be provided in further combination with a magnetic field emitting element. In this particular example, one or more laser diodes and one or more light emitting diodes are used in conjunction with a magnet or magnetic field emitting element, and all elements being incorporated within the flexible member of the devices.

For certain applications, it is most preferred to provide a device having a previously described flexible sealing member in combination with a laser light emitting element and a light emitting diode (LED). In other applications, it is most preferred to provide a device with a previously described flexible sealing member in combination with either one of the laser light emitting elements or the light emitting diode (LED). Preferred wavelengths of light emitted from the laser and the LED range from 600 nm to 910 nm.

The preferred embodiment device comprising the flexible sealing member and the one or more energy emitting elements, is in certain versions, adapted to be connected to a base component of the therapeutic instrument or device. Typically, the preferred embodiment device can use a standard electronic cable to provide the requisite electrical power and electronic communication between the base component and the device. It is also contemplated that the cable could be in the form of one or more optic cables that transmit light from the base device to the flexible sealing members. For this configuration, the one or more light emitting elements could then be incorporated in the base component. Alternatively, for other preferred embodiments, the device comprising the flexible sealing member and the one or more energy emitting elements, can also comprise the power and control components such that the entire device is a one-piece device, and so no cable connection is necessary.

The preferred embodiment device is used by selecting a region of a person's body that is to be treated. That region should be exposed and free from clothing or other interfering garments or items such as jewelry. The device is placed over the region of interest, and particularly such that the face of the sealing member is directly over the region of interest. Preferably, the distal edge of the wall, upon contacting the skin, extends entirely around the affected area or treatment region. The device may be slightly urged against the user's skin to promote a sealing engagement between the skin and the distal edge of the wall. In certain instances, it is contemplated that the seal may be improved by wetting that region, or by the use of certain substances such as rubbing alcohol, oil, water, or even a user's own saliva. Upon establishing a seal, the flexible sealing member is then compressed to evacuate a portion of the air within the interior region of the sealing member. That air or contents of the interior of the sealing member may be passed between the skin and distal edge of the wall, thereby temporarily breaking the previous seal, or the air may exit through one or more ports defined in the device, such as for example in the form of one-way valves disposed within the sealing member. After evacuation of a portion of the previous contents of the sealing member, the elastic and semi-rigid characteristic of the flexible sealing member causes the member to revert back to its original shape, and thus, original volume. As will be appreciated, that causes the creation of a vacuum within the interior region of the sealing member. The extent of the previous evacuation and the change in volume of the sealing member (if it was previously deformed), determine the extent of vacuum created within the sealing member. As will be appreciated, the device or rather the flexible sealing member can be removed simply by removing the vacuum, such as by breaking the seal along the distal edge. Means may be used to selectively control the amount of vacuum established within the flexible member. For instance, it is contemplated to place the interior region of the flexible member in communication with a conduit and vacuum pump, whereby a desired degree of vacuum could be attained within the member. And thus, this latter strategy could be used to achieve a vacuum between the member and the skin in contact therewith, without resort to flexing and compressing the member to evacuate a portion of its contents.

As previously noted, the present invention device, in certain embodiments, is utilized to promote the accumulation and/or concentration of blood in a body region of interest by application of a vacuum to the body region. This practice enables the blood within that region to receive the therapeutic effects of the one or more energy emitting elements in the present invention devices. A specific example of this, is in the irradiation of blood by exposure to the emitted energy. As previously noted, a preferred technique is the irradiation of the accumulated or concentrated blood within the body region of interest, by exposure to one or more specific ranges of wavelengths of light, such as ultraviolet light, infrared light, and/or light within the visible region having a specific color. Another preferred form of emission for irradiating blood, is a magnetic field. However, it is to be understood that the present invention includes a wide array of other irradiation strategies.

More specifically, by use of the present invention devices and strategies, methods for intensifying blood circulation are provided. In a further aspect according to the present invention, upon attaining or while concurrently attaining, an increased state of blood circulation by creating a vacuum with the flexible member of the present invention device, the portion of blood that is within, proximate, or adjacent the epidermis, dermis and/or the subcutaneous layer, is irradiated with one or more forms of energy emitted from the energy emitting element(s). However, benefits result from solely increasing blood circulation by use of the present invention to create a vacuum over the body region of interest. And, further benefits can result from increasing blood circulation in conjunction with irradiation by use of the present invention. Increases in blood circulation are measurable and so increases in such can be quantified. Methods based upon changes in skin temperature, thermal conductance, volume plethysmography and light absorption of the skin are known in the art for measurement of cutaneous circulation, see Wright, “Measurement of the Cutaneous Circulation,” J. Appl Physiol 20: 696-702, 1965. By use of the present invention, increases in blood circulation within the dermal region, i.e. including the epidermis, dermis, and/or hypodermis, are increased by at least 10%, more preferably by 20%, more preferably by 30%, more preferably by 40%, and most preferably by 50% or more. In certain instances, after creation and maintenance of a vacuum by use of the flexible sealing member, blood circulation in the region proximate the sealing member is increased many times. For example, increased capillary flow such as from 10 to 15 times, has been achieved by use of the preferred embodiment flexible sealing members. These increases in circulation can be achieved by creating and maintaining a vacuum over the body region of interest, by use of the present invention. It is particularly preferred to administer one or more forms of energy emission while having attained an increased state of blood circulation. Preferred combinations of energy emission and elevated states of blood circulation are as follows, in Table 2 below.

TABLE 2
Combinations of Intensified Blood Circulation and Irradiation

| | Increased Blood Circulation | | | | |
|---|---|---|---|---|---|
| | 10% | 20% | 30% | 40% | 50% or more |
| Electromagnetic radiation | x | x | x | x | x |
| laser light, | x | x | x | x | x |
| pulsed light, | x | x | x | x | x |
| ultraviolet | x | x | x | x | x |
| infrared, | x | x | x | x | x |
| specific colors (e.g. blue, | x | x | x | x | x |
| red, etc) | | | | | |
| Thermal energy (heat) | x | x | x | x | x |
| Magnetic fields | x | x | x | x | x |
| Electrical fields and current | x | x | x | x | x |
| (TENS) | | | | | |

Although not wishing to be limited to any particular theory, it is believed that increasing blood circulation in a body region of interest is beneficial since greater amounts of blood may then be exposed to laser and/or infrared radiation (or other forms of energy as described herein). After such exposure, processed and/or irradiated blood having improved biological, biochemical, and/or immunological properties, enters other tissue regions. At these other regions, blood exchange occurs thereby imparting anti-inflammatory, pain-releasing, anti-edema, antitoxic, and/or relaxing effects. This leads to rapid restoration of normal physiological conditions of tissue.

In addition, in certain applications, upon creation and maintenance of a vacuum by use of the preferred embodiment sealing members, old or brittle capillaries that do not participate in metabolic and biological exchange processes may rupture. This may be beneficial. Unlike main blood vessels, the ability of capillaries to regenerate leads to renewed microcirculation and normalization of local cellular metabolism.

It is particularly preferred to utilize a specific combination of emissions, or at least one or more of these emissions as follows. Pulsed infrared laser radiation at a wavelength of about 905 nm penetrates tissue at a depth of about 10 cm to about 13 cm, and has strong stimulating effects on blood circulation membranes and intracellular metabolism. Laser emits energy which is absorbed by cells and tissue resulting in conversion of light into biochemical energy. Multiple physiological responses are thereby initiated and normal cell function can be restored. This process is curative and can eliminate many symptoms. Pulsed infrared radiation at a wavelength of about 875 nm penetrates shallower tissue depth and has an overall broader spectrum as compared to laser radiation. Red light at a wavelength of about 660 nm penetrates smaller tissue depth and has beneficial anti-inflammatory effects. Emission of a magnetic field keeps ionized molecules of tissue in a disassociated stage, thus enhancing the energy potential at the molecular and cellular levels. The application of a magnetic field is also believed to provide energy-mediated protection of organisms against environmental impacts such as climatic factors and electromagnetic fields.

Generally, overall emission power levels range from about only several milliwatts to about 500 mW, per element. Peak pulse power levels of up to 25,000 mW with an average power of about 7.5 mW are generally preferred as such power levels can achieve high depth of tissue penetration while providing gentle average power levels. It will be appreciated that continuous power levels greater than about 500 mW are generally not preferred since such can damage tissue. By pulsing the energy emitting element, significantly higher power levels can be used, so long as the mean output power level is less than about 500 mW.

The present invention devices and methods can be used to treat or assist in the treatment of a wide range of ailments, maladies, and conditions. Examples of these include, but are not limited to, various pain relief solutions such as acute pain relief, subacute and chronic pain relief, shoulder pain, carpal tunnel, back pain, tennis elbow, arthritic pain, fibromyalgia and many other pain indications; anti-aging and cosmetics solutions such as those relating to varicose veins, cellulite, acne, wrinkles, psoriasis, and other cosmetic indications; wound management solutions such as tissue repair and cell regeneration, skin ulcers, diabetic ulcers, burns, postoperative wound care, and other wound-associated indications; spinal diseases such as osteochondriosis, scoliosis, intervertebral disk hernia, post-traumatic pain, carriage disorders, radiculitis, ischialgia, joint diseases such as arthritis, arthrosis, coxarthrosis, contractures, calcaneal spurs, myositis; and other conditions such as sunburns, acute (traumatic) & chronic pain, neck & back pains, tendonitis, golfer elbow, eczema, non-healing ulcers, sinusitis, tonsillitis, fibromyalgia, migraines, tinnitus, and similar conditions and ailments.

Specifically, using the present invention devices to thereby create and maintain a vacuum upon a tissue region of interest while exposing such tissue to particular energy as described herein can provide treatment regimes and relief with regard to a variety of disorders such as, but not limited to, cellulite, obesity, arthritis, arthrosis, epicondilitis, joint injuries occurring during trauma, post bone fractures, osteochodritis of the vertebrae, deformed spondylitis, spondilyarthrosis, shoulder and scapular arthritis, acne, particularly in the back and chest regions, osteoporosis, myositis, and osteomyelitis.

Using the preferred embodiment flexible sealing member with the devices and methods described herein may also be beneficial for treating disorders associated with rupturing of skin, skin disorders, mainly of infectious etiology, inflammatory disorders of skin, particularly those involving discharge of pus which is often produced during inflammatory responses of the body, and disorders associated with increased rupturing of vessels and decreased coagulation. Additional disorders for which the present invention can be applied, include varicose veins in extremities, burns and frost bites, diabetes, high fever, severe pain syndrome during acute disease or trauma, or hemorrhages of any etiology.

Many other benefits will no doubt become apparent from future application and development of this technology.

It will be appreciated that the present invention devices include combinations of all of the various energy emitting elements, and combinations of such in conjunction with the noted strategies of creating a vacuum along a body region of interest, and further in optional combination with an irradiating procedure. Any of the various features of one embodiment may be combined or used with one or more other feature(s) of other embodiments.

All referenced patents and documents, are hereby incorporated herein in their entirety.

As described hereinabove, the present invention solves many problems associated with previous type devices. However, it will be appreciated that various changes in the details, materials and arrangements of parts, which have been herein described and illustrated in order to explain the nature of the invention, may be made by those skilled in the art without departing from the principle and scope of the invention, as expressed in the appended claims.

What is claimed is:

1. A non-invasive therapeutic device adapted for selective attachment to and removal from a surface region of an individual's body, the device comprising:
a flexible member defining a medial portion and a wall extending outwardly therefrom, the wall and the medial portion defining a hollow interior accessible from a face of the flexible member, the wall defining a distal edge that upon attachment of the device to the body, sealingly contacts the surface region of the body, the medial portion and the wall both defining an inner surface facing the hollow interior of the flexible member; and
at least one element adapted to emit a therapeutically effective level of energy, the element affixed to the inner surface of the flexible member.

2. The therapeutic device of claim 1, wherein upon attachment of the device to the surface region of an individual's body, the flexible member serves to intensify blood circulation in the body region at which the device is attached.

3. The therapeutic device of claim 1, wherein the at least one element is selected from the group consisting of (i) a light emitting element, (ii) a heat emitting element, (iii) a magnetic field emitting element, (iv) an electrical field emitting element, (v) an electrical current emitting element, and (vi) combinations thereof.

4. The therapeutic device of claim 3, wherein the at least one element includes two different elements selected from (i)-(v).

5. The therapeutic device of claim 3, wherein the at least one element includes a plurality of light emitting elements.

6. The therapeutic device of claim 5, wherein the at least one element includes a plurality of light emitting diodes and a laser emitting element.

7. The therapeutic device of claim 3 wherein the at least one element includes a light emitting element that emits light at a wavelength range selected from the group consisting of (i) 8,000-15,000 nm, (ii) 3,000-8,000 nm, (iii) 1,400-3,000 nm, (iv) 750-1,400 nm, (v) 620-750 nm, (vi) 590-620 nm, (vii) 570-590 nm, (viii) 495-570 nm, (ix) 450-495 nm, (x) 380-450 nm, (xi) 200-400 nm, (xii) 320-400 nm, (xiii) 280-320 nm, (ixv) 10-280, (xv) 10-200, and (xv) combinations thereof.

8. The therapeutic device of claim 7 wherein the at least one element includes a light emitting element that emits light in a pulse fashion.

9. The therapeutic device of claim 7 wherein the at least one element includes a light emitting element that emits laser light.

10. The therapeutic device of claim 3 wherein the at least one element is an electrical current emitting element and the device further comprises at least one electrode.

11. The therapeutic device of claim 1 wherein the at least one element is a light emitting element that emits light having a wavelength in the range of 600 nm to 910 nm.

12. The therapeutic device of claim 11 wherein the light emitting element is selected from the group consisting of (i) a laser light emitting element, (ii) a light emitting diode, and (iii) combinations of (i) and (ii).

13. A method for selectively attaching a non-invasive therapeutic device to a surface region of an individual's body, the method comprising:
providing a device including (i) a flexible member defining a medial portion and a wall extending outwardly from the medial portion, the wall and the medial portion defining a hollow interior accessible from a face of the flexible member, the wall defining a distal edge, and (ii) at least one element disposed in and affixed to the flexible member, and adapted to emit a therapeutically effective level of energy; and
contacting the distal edge of the wall of the flexible member of the device to the surface region of the body; and
creating a vacuum within the hollow interior, thereby attaching the device to the surface region of the body.

14. The method of claim 13, wherein the vacuum is created by temporarily compressing the flexible member, sealingly contacting the distal edge to the surface region of the body, and allowing the flexible member to return to its previous shape prior to compressing.

15. The method of claim 13, wherein the vacuum is created by placing the hollow interior of the flexible member in communication with a vacuum-producing means, and creating a vacuum therein.

16. A method for intensifying blood circulation in a body region while concurrently applying a therapeutically effective level of energy to that region, the method comprising:
providing a non-invasive therapeutic device adapted for selective attachment to and removal from a surface region of an individual's body, the device comprising (i) a flexible member defining a medial portion and a wall extending outwardly therefrom, the wall and the medial portion defining a hollow interior accessible from a face of the flexible member, the wall defining a distal edge that upon attachment of the device to the body region, sealingly contacts the surface region, the medial portion and the wall both defining an inner surface facing the hollow interior of the flexible member, and (ii) at least one element adapted to emit a therapeutically effective level of energy, the element affixed to the inner surface of the flexible member;
attaching the device to a surface region of an individual's body by sealingly contacting the distal edge of the wall to the surface region and creating a vacuum in the hollow interior of the flexible member, thereby intensifying blood circulation in the body region by at least 10%; and
emitting a therapeutic level of energy to the body region while the device is attached to the body region.

17. The method of claim 16 wherein the emitted energy is energy emitted from an element selected from the group consisting of (i) a light emitting element, (ii) a heat emitting element, (iii) a magnetic field emitting element, (iv) an electrical field emitting element, (v) an electrical current emitting element, and (vi) combinations thereof.

18. The method of claim 17, wherein the at least one element includes two different elements selected from (i)-(v).

19. The method of claim 16, wherein the at least one element includes a light emitting element.

20. The method of claim 16, wherein the at least one element includes a plurality of light emitting diodes and a laser light emitting element.

21. The method of claim 16 wherein the at least one element includes a light emitting element that emits light at a wavelength in the range of 600 nm to 910 nm.

22. The method of claim 21 wherein the at least one element includes a light emitting element that emits light in a pulse fashion.
